(19) United States
(12) Patent Application Publication
(10) Pub. No.: US 2007/0294104 A1
(43) Pub. Date: Dec. 20, 2007

(54) System, Method, and Means, to Detect and Prevent Fraudulent Medical Insurance Claims

(76) Inventors: Carmeli Boaz, Koranit (IL); James H. Kaufman, San Jose, CA (US); David C. Spellmeyer, Oakland, CA (US)

Correspondence Address: FREDERICK W. GIBB, III;Gibb & Rahman, LLC, 2568-A RIVA ROAD, SUITE 304, ANNAPOLIS, MD, 21401, US

(21) Appl. No.: 11/424,329

(22) Filed: Jun. 15, 2006

(51) Int. Cl. G06Q 10/00; A61B 5/00

(52) U.S. Cl. 705/2; 600/300

(57) ABSTRACT

The invention provides a method for detecting and preventing fraudulent medical insurance claims comprising storing identifying information and medical transaction histories on a portable device and on a server. The portable device is presented by a valid patient user to an authorized medical care provider. Medical transaction histories stored transaction histories are detected from the portable device and server. Medical transactions histories stored on the device are compared with those stored on the server. When the transaction histories on each device do not match, potential fraudulent medical insurance claims are detected.

### BACKGROUND

1. Field of the Invention

The embodiments of the invention generally relate to health care fraud detection, and, more particularly, to a method and system for detecting fraudulent medical insurance claims by providing a patient user with a portable device storing the user's identification information and the user's medical history. Portable devices may include, for example, a smart card, a flash memory device, a magnetic strip card, etc. A remotely located server also stores the information and history. Potential medical fraud may be identified by detecting inconsistencies in the information and histories stored on the server and portable device.

2. Description of the Related Art

In the health care industry electronic means for storing medical information include electronically storing data on a mainframe computer, computer server or LAN within a medical provider's locality, for example, hospital, physician's office, laboratory, pharmacy, etc. Because the information is stored locally, there exists a risk of fraud. If a patent's information is stolen or modified from one provider and used elsewhere to obtain medication, for example, it is very difficult to adequately identify that any fraud has occurred. Another risk inherent in such systems is miscommunication, misunderstanding or lack of knowledge of past medical problems and procedures by the varied medical care providers storing patient data and medical histories. The health care providers can make diagnoses and prescribe treatment with less than the totality of the patient's medical and treatment history.

Fraud and abuse may take place at many points in the health care system. doctors, hospitals, nursing homes, and diagnostic facilities, for example. Health care is especially susceptible to electronic data interchange fraud. Electronic data interchange (EDI) is the direct filing of claims, computer to computer, and is widely used for Medicare claims. Thus, given the growing number of claims sent electronically, improved methods for identifying and preventing fraud particularly in the health care system are needed.

### SUMMARY

In view of the foregoing, an embodiment of the invention provides a method for detecting and preventing fraudulent medical insurance claims comprising storing identifying information and medical transaction histories on a portable device and on a server. The portable device is presented by a valid user, e.g. patient, to an authorized entity, e.g. physician. Stored medical transaction histories are detected from the portable device and server. Medical transactions histories stored on the patient's device are compared with those stored on the server. When the transaction histories on each device do not match, fraudulent inconsistencies, e.g. potential fraudulent insurance claims, are detected.

In another aspect of the invention, the medical transaction histories of a patient user are compiled from a plurality of sources. A patient may be covered by multiple insurance policies and visit various medical care providers. Therefore, it is advantageous that a system and method be able to compile information from many sources. Moreover, individuals see various medical care providers for their health care needs. In this method, the information is collected from the various medical care providers, for example, primary care physicians, specialists, laboratories, pharmacies, etc. Thus a complete medical transaction history is stored in both the portable storage device and the server.

In yet another aspect of the invention, the patient user presents the portable storage device to a medical care provider who downloads the information and histories from both the portable device and server. When the information and histories are compared. If the information and histories match, the medical transaction histories on both the portable device and the server are updated to include the most recent medical transaction.

These and other aspects of the embodiments of the invention will be better appreciated and understood when considered in conjunction with the following description and the accompanying drawings. It should be understood, however, that the following descriptions, while indicating preferred embodiments of the invention and numerous specific details thereof, are given by way of illustration and not of limitation. Many changes and modifications may be made within the scope of the embodiments of the invention without departing from the spirit thereof, and the embodiments of the invention include all such modifications.

### BRIEF DESCRIPTION OF THE DRAWINGS

The embodiments of the invention will be better understood from the following detailed description with reference to the drawings, in which:

FIG. 1 is a flow diagram illustrating a preferred method of an embodiment of the invention;

FIG. 2 illustrates a schematic diagram of a hardware system for the practice of the present invention; and

FIG. 3 illustrates a schematic diagram of a system for the practice of the present invention.

### DETAILED DESCRIPTION OF PREFERRED EMBODIMENTS

The embodiments of the invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the embodiments of the invention. The examples used herein are intended merely to facilitate an understanding of ways in which the embodiments of the invention may be practiced and to further enable those of skill in the art to practice the embodiments of the invention. Accordingly, the examples should not be construed as limiting the scope of the embodiments of the invention.

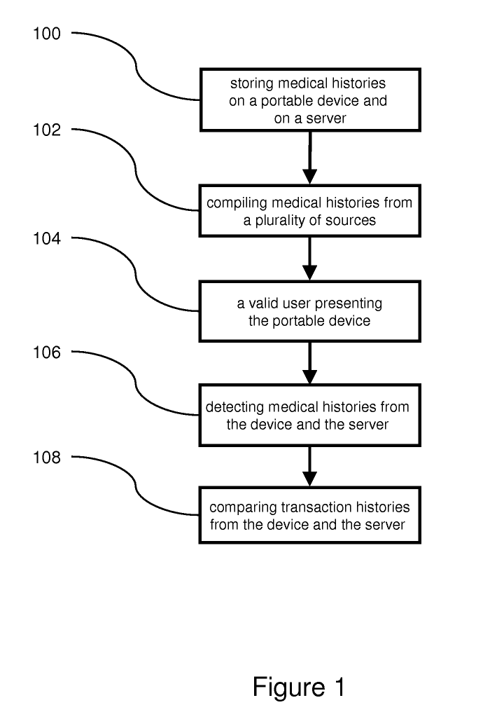

As mentioned, there remains a need for a method and system for detecting medical insurance fraud. The embodiments of the invention achieve this by providing a method for detecting medical insurance and health care fraud. Referring now to the drawings, and more particularly to FIGS. 1 through 3, where similar reference characters denote corresponding features consistently throughout the figures, there are shown preferred embodiments of the invention.

The high cost of medical care and the low rates of insurance coverage have set the conditions for widespread insurance fraud in medical care. Furthermore, the forthcoming adoption of modern information technology applied to electronic health records and payments may accelerate the threat of insurance fraud. Fraud and abuse take place at many points in the health care system including doctors, hospitals, nursing homes, diagnostic facilities and pharmacies, for example. Health care is especially susceptible to electronic data interchange fraud. Thus, implementation of methods to identify and prevent medical insurance fraud will help to keep health insurance costs down and protect a patient's medical history and privacy.

As the U.S. and other countries around the world move to modern electronic health records, the ability of criminals to steal or otherwise misappropriate insured patient identities, and therefore, to make fraudulent claims against the patient's insurance policies will become an increasing threat. The efficiency of electronic information systems vastly increases the rate at which this type of fraud can cause monetary including damage to the patient's identity and health record itself. The present invention uses the patient's own medical information to protect against such fraud.

By its very nature, healthcare, and therefore electronic health records is distributes. A modern information system for managing health records in the U.S. will require that patients have access to a registry of metadata that indirectly points to the locations where their records are stored. With the invention, this registry is stored on a smart card, a Universal Serial Bus (USB) Key, a Personal Computer (PC), magnetic strip card, or other pervasive device. The data could also be stored on-line in the patient's independent account at a health record bank. Any time the patient goes to a medical care provider for services, the medical care provider can access some parts of this data to support the continuity of patient care. The provider also can access the patient's identification and medical history for billing and payment purposes.

Identity theft and stolen insurance information will become vastly more damaging once electronic health records and payment systems become ubiquitous. Furthermore, the large number of uninsured in our society may cause some, out of desperation, to use counterfeit health insurance cards or use stolen insurance information to obtain care; thus, passing the cost of theft on to the insurance companies, the providers and other insured patients.

Unlike fraud involving financial systems, medical insurance fraud can take place based on the fact that individuals hold more than one insurance policy and visits various medical care providers. In medical fraud there is a more complex range of preventable crimes based on use of the coded entities in a patient's health record. The range of medical fraud that may be prevented includes: charging the same treatment to multiple policies; charging for incompatible treatments; using multiple policies and multiple providers to obtain drugs that may, for example, then be sold; and identity theft across policies.

In financial systems, one does not see data or information being added to a particular individual's record. In other words, the victim of fraud would not see information, i.e. money, added to his bank or credit card account. Generally, in credit card or bank fraud, the victim sees money removed from his account. In health records, data may be added or removed in order to perpetuate fraud of an individual or of a company without the victim's knowledge. For instance, fraudulent deposition of a misdiagnosis could be used to gain access to healthcare or medications. For example, one having fraudulent intent could add a record that he has chronic back pain and must be prescribed a potent pain medication. The individual could then obtain a prescription for that medication based on the fraudulent record one in which data has been deposited. Another example would be someone with a diagnosis that would preclude some sort of health benefit or treatment. In such a situation, an individual might want to “deposit” a second diagnosis that contradicts the first or remove the unwanted report. Thus, in health care fraud, the complexity of the health care system makes the detection of fraud a challenge.

FIG. 1 illustrates a flow diagram according to an embodiment of the invention for a method of detecting and preventing medical insurance fraud of the invention.

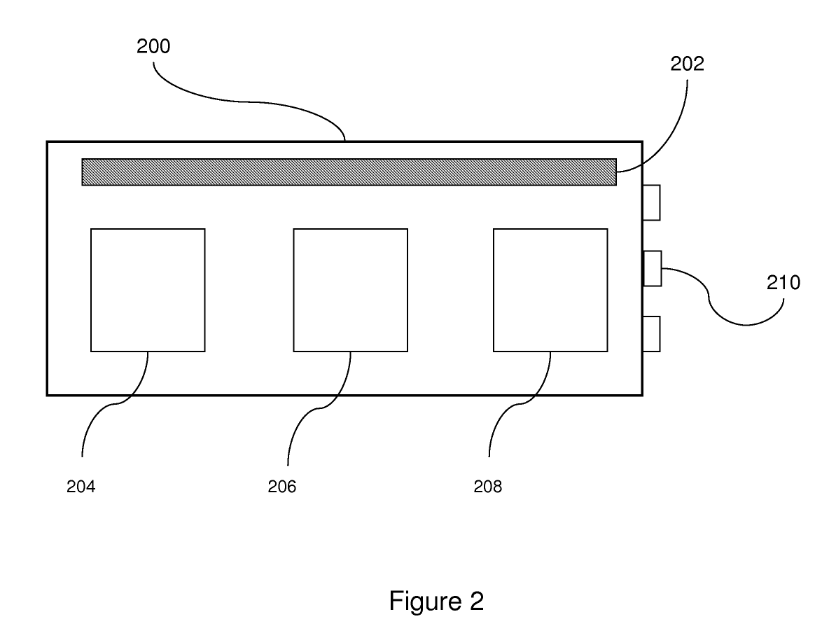

The method detects and prevents fraudulent medical insurance claims by storing identifying information and medical transaction histories on both a portable storage device and on a server (100). The portable storage device (200 in FIG. 2) of the method may comprise, for example a smartcard, a USB key, a magnetic strip card, a PC, personal digital assistant (PDA), cell phone, or even an online service owned by the patient as well as any other similar portable device. This portable storage device comprises a registry of patient identifying information and medical histories. The information could be just a reference to the documents by universal resource locator (URL). The patient identifying information and medical history also resides on a server (202) accessible directly by the patient's portable device, by the medical care provider or by the insurance carrier. U.S. Pat. No. 5,832,488, incorporated herein by reference, discloses a system and method for programming the storage of medical histories on a storage device and a second computer. U.S. Pat. No. 5,832,488 provides a way to automatically collect data for the purposes of providing the patient and attending physician a electronic health record to be used in patient care. The invention described herein takes advantage of the historical data within that record to detect anomalies like identity theft and to prevent fraud.

The stored information and histories on the device and the server are compiled from a plurality of sources (102). Those sources include various medical care providers including, but not limited to, primary care physicians, specialists, pharmacies, hospitals and laboratories. A patient user presents the portable device to a medical care provider at the time of receiving medical services, medical supplies, medicines, etc. (104). When the patient presents the portable device, the stored information and medical history stored on the server is detected (read) (106).

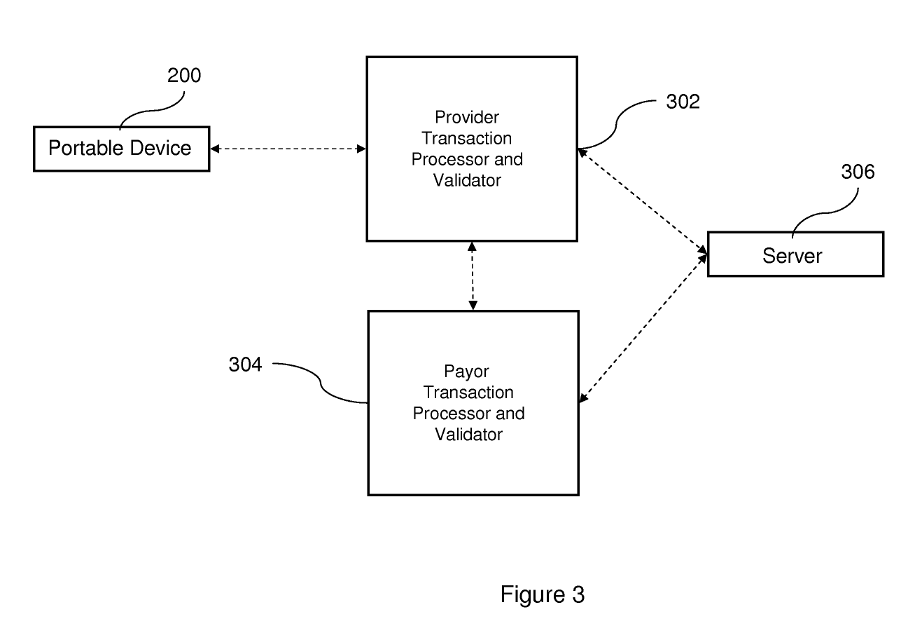

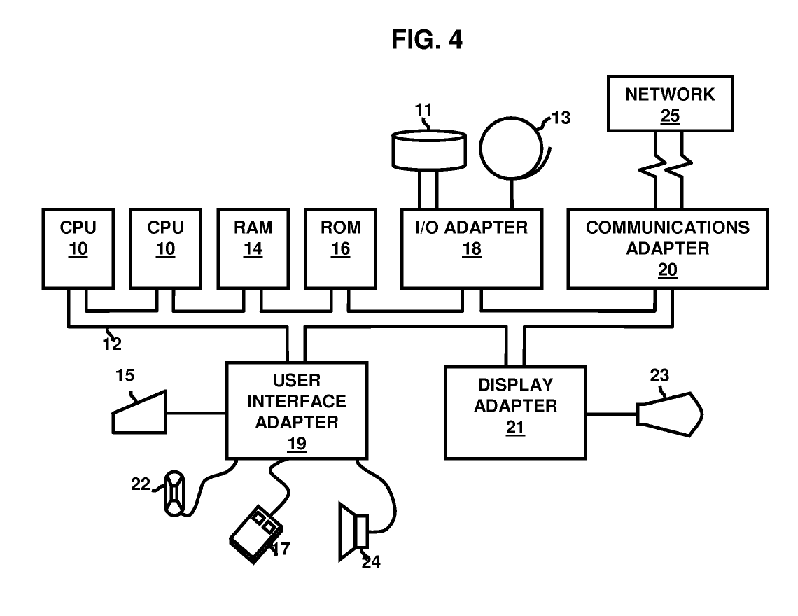

The information and medical histories on the portable storage device presented by the patient can be handled by a healthcare provider transaction processor and validator. As shown in FIG. 3, the healthcare provider transaction processor and validator (302) is the application used by the provider for billing and validation of patient data. This application runs on a PC and downloads the data from the patient's portable device. The payor transaction processor and validator (304) contact the payor's (insurance provider's) system to validate patient information. The payor's side system communicates with the server that has the patient's compiled medical history so that the information downloaded from the portable device by the provider's validator and the information from the server can be compared. Inconsistencies trigger a fraud alert and payment would not be approved. The information and medical transaction histories stored on both devices are compared for inconsistencies or irregularities (108).

When the information on the portable device show inconsistencies of either added or deleted data, potential medical insurance fraud is indicated by the inconsistent data. An alert to potential fraud is triggered on the server and/or portable device. Additionally, the alert is also beneficial in circumstances where inconsistencies in drug dosage are noted by detecting inconsistencies in the records, for example. The detection of the inconsistency, in addition to preventing fraud, alerts the medical care provider to potential medical errors.

In circumstances were the information and medical histories on both the portable storage device and the server are the same or consistent with each other, the information and medical histories on each device are updated to include the current transaction the patient is requesting on presentation of the portable storage device. The provider accesses the patient's information and medical history on the portable storage device present at the time of service. The payor matches the local history from the patient's portable device with the history data on the server. If the information and histories on the patient's device and the information from the server accessed by the provider's transaction processor and validator match, the transaction is considered valid. The provider approves the treatment and sends a message, e.g. a bill, which updates the payor's registry and also the server. That information is communicated to the server and the portable device to update the patient's identifying information and medical histories on each device. Both the provider's process validator and the payor's process validator communicate with the server to access the patient information and medical history because the system may detect medical fraud even in instances where insurance is not involved.

In an example of a fraudulent encounter, a criminal organization or individual steals or otherwise misappropriates the patient's identifying information and medical history or makes a counterfeit copy of the patient's portable storage device. A copy of the patient's device is made. The copied device is presented to a provider. The provider accesses the patient's identifying information and medical history from the device and the server. The provider or payor matches the local history from the patient's portable device with the history data on the server. Data from the copy is considered valid, so provider approves treatment. The provider sends a message, e.g. a bill, that updates the server and the patient's personal registry on the copied device with a reference to the new encounter with a medical provider for services. The data on the counterfeit device is now out of synch with the data on the actual patient's own copy of his medical history on his portable device. The very next time the patient sees a provider the provider and payor system will discover the synchronization error and inform the patient of the possible fraud. If multiple counterfeit copies are used, the first time a single counterfeit device is used all counterfeit devices become out of synch. Thus, the fraud is contained to the use of the illegal counterfeit use of a single copy of the patient's information and history from his device. The fraud is automatically detected any time a second a device is used. The more widespread the counterfeit, the faster the fraud is detected with the present method.

Fraud can be detected even more quickly than described above using direct messaging between the patient's device and the server. With each encounter for medical services the patient can be notified electronically of the new encounter and thus be informed on the very first fraudulent use or billing to a health insurance policy.

The embodiments of the invention can take the form of an entirely hardware embodiment, an entirely software embodiment or an embodiment including both hardware and software elements. In a preferred embodiment, the invention is implemented in software, which includes but is not limited to firmware, resident software, microcode, etc.

Furthermore, the embodiments of the invention can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments of the invention is depicted in FIG. 2. This schematic drawing illustrates a simplified hardware configuration of the above-described portable storage device (200) comprising, for example, a magnetic strip (202), a re-writable memory device (204) patent for storing patient identifying information and medical histories, a central processing unit (CPU) which may include a display device, computer, etc. (206), a power supply (208), and connectors (210) to connect the device to a reader. As would be understood by those ordinarily skilled in the art in light of this disclosure, FIG. 2 illustrates only one example of the portable device. The portable device could comprise all elements of any well-known device such as a smart card, PDA, PC, USB device, cell phone, etc., the details of which are omitted herefrom to focus the reader on the salient aspects of the invention. Therefore, for example, the memory 204 could comprise any form of re-writable memory including magnetic media, hard drive, flash memory, etc. Similarly, the CPU/display 206 can comprise any form of processor and display from a simple logic circuit and light emitting diodes to a system-on-a-chip with an active matrix display. For example, see U.S. Pat. No. 6,798,647, U.S. Pat. No. 7,039,759, and U.S. Pat. No. 7,025,277, which are incorporated herein by reference.

FIG. 3 represents a representative hardware configuration comprising the above-described portable storage device (200), provider transaction processor and validator (302), payor transaction processor and validator (portable device reader) (304), and server (306).

A representative hardware environment for practicing the embodiments of the invention is depicted in FIG. 4. This schematic drawing illustrates a hardware configuration of an information handling/computer system in accordance with the embodiments of the invention. The system comprises at least one processor or central processing unit (CPU) 10. The CPUs 10 are interconnected via system bus 12 to various devices such as a random access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system can read the inventive instructions on the program storage devices and follow these instructions to execute the methodology of the embodiments of the invention. The system further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network 25, and a display adapter 21 connects the bus 12 to a display device 23 which may be embodied as an output device such as a monitor, printer, or transmitter, for example.

The foregoing description of the specific embodiments will so fully reveal the general nature of the invention that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments of the invention have been described in terms of preferred embodiments, those skilled in the art will recognize that the embodiments of the invention can be practiced with modification within the spirit and scope of the appended claims.

What is claimed is:

1. A system for detecting medical record inconsistencies comprising:
a portable device adapted to be carried by a patient and to store identifying information and medical transaction histories;
a server adapted to store said identifying information and medical transaction histories;
a portable device reader connected to said server, wherein said portable device is adapted to temporarily connect to said portable device reader; and
a comparator connected to said server and adapted to compare said identifying information and said transaction histories stored on said portable device and stored on said server.

2. The system of claim 1, further comprising a transaction process validator connected to said portable device and said server.

3. The system of claim 1, further comprising a compiler connected to said server and adapted to compile said identifying information and said medical transaction histories from a plurality of medical insurance policies, and medical care providers.

4. The system of claim 1, wherein said server is adapted to update said identifying information and said medical transaction histories on said portable device and said server when said identifying information and said medical transaction histories on said portable device and said server are consistent with each other.

5. The system of claim 1, said server is adapted to not update said identifying information and said medical transaction histories on said portable device and wherein said server when said identifying information and said medical transaction history on said portable device and said server are not consistent.

6. The system of claim 1, wherein said server is adapted to produce an alert on said portable device and said server when said identifying information and said medical transaction history on said portable device do not match said identifying information and said transaction histories stored on said server.

7. An apparatus for detecting medical record inconsistencies comprising:
a portable device adapted to be carried by a patient and to store identifying information and medical transaction histories in a rewritable electronic memory,
wherein said portable device is adapted to connect to a system comprising:
a server adapted to store said identifying information and medical transaction histories;
a portable device reader connected to said server, wherein said portable device is adapted to temporarily connect to said portable device reader; and
a comparator connected to said server adapted to compare said identifying information and said transaction histories stored on said portable device and stored on said server.

8. The apparatus of claim 7, wherein said connector comprise at least one of a physical connection and a wireless connection.

9. The apparatus of claim 7, further comprising a compiler connected to said server and adapted to compile said identifying information and said medical transaction histories from a plurality of medical insurance policies, and medical care providers.

10. The apparatus of claim 7, wherein a transaction processor and validator are connected to said portable device and said server.

11. The apparatus of claim 7, wherein said portable device and said server are adapted to compile said identifying information and said transaction histories from a plurality of medical policies and a plurality of medical care providers.

12. The system of claim 7, wherein said server is adapted to update said identifying information and said medical transaction histories on said portable device and said server when said identifying information and said medical transaction histories on said portable device and said server are consistent with each other.

13. The system of claim 7, wherein said server is adapted to not update said identifying information and said medical transaction histories on said portable device and said server when said identifying information and said medical transaction history on said portable device and said server are not consistent.

14. The system of claim 13, wherein said server is adapted to produce an alert on said portable device and said server when said identifying information and said medical transaction history on said portable device do not match said identifying information and said transaction histories stored on said server.

15. A method for detecting and preventing fraudulent medical insurance claims comprising:
storing medical transaction histories on a portable device and a server;
comparing said medical transaction histories on said portable device with said medical transaction histories on said server; and
responsively noting inconsistencies between said medical transaction histories on said portable device and said server.

16. The method of claim 15, wherein said inconsistencies indicates one selected from: fraudulent inconsistencies and erroneous inconsistencies.

17. The method of claim 16, wherein said fraudulent inconsistencies in said medical transaction histories includes at least one selected from the group of: added data and deleted data.

18. The method of claim 15, wherein said transaction histories are complied from multiple medical insurance policies or multiple medical service providers.

19. The method of claim 16, wherein said fraudulent inconsistencies are detected when a valid user attempts to update said medical transaction histories after an unauthorized user has updated said medical transaction histories and said medical transaction histories on said portable device does not match said medical transaction histories on said server.

20. The method of claim 16, wherein said erroneous inconsistencies are detected when a valid user attempts to update said the medical transaction histories and said medical transaction histories on said portable device and said medical transaction histories on said server do not match.
